(19) United States
(12) Patent Application Publication
(10) Pub. No.: US 2026/0167610 A1
(43) Pub. Date: Jun. 18, 2026

(54) CLEAN PRODUCTION METHOD FOR EPOXYPROPANE USING THE CHLOROHYDRINATION PROCESS WITH A CHLORINE CLOSE-LOOP CIRCULATION

(71) Applicant: China University of Petroleum (East China), Qingdao (CN)

(72) Inventors: Yuanyu TIAN, Qingdao (CN); Yingyun QIAO, Qingdao (CN); Yiliang TIAN, Qingdao (CN); Peijie ZONG, Qingdao (CN); Jingxian WANG, Qingdao (CN); Wenlong XU, Qingdao (CN); Wenshuo MA, Qingdao (CN); Yanpeng ZHANG, Qingdao (CN); Ruotong CUI, Qingdao (CN); Tingshuai TIAN, Qingdao (CN)

(21) Appl. No.: 19/421,463

(22) Filed: Dec. 16, 2025

(30) Foreign Application Priority Data
Dec. 17, 2024 (CN) 202411865897.4

(51) Int. Cl. C07D 301/32

(52) U.S. Cl. CPC C07D 301/32

(57) ABSTRACT

The disclosure relates to a clean production method for epoxypropane using a chlorohydrination process with chlorine closed-loop circulation, comprising:
(1) Chlorohydrination: dissolving chlorine in water, feeding into a reactor to generate chloropropanol solution, washing tail gas with NaOH electrolyte, and returning to propylene recovery;
(2) Causticization: reacting chloropropanol solution with NaOH, feeding into a stripping tower to obtain crude epoxypropane and causticization wastewater;
(3) Rectification: crude epoxypropane passes through a front distillation tower, epoxypropane extracted from the top, high-boiling components discharged from fractionating tower, both under normal pressure;
(4) Wastewater treatment: supercritical gasification and separation of stripping tower wastewater, tail gas washing liquid, and fractionating tower wastewater, diluting NaCl slurry and returning to chlorine-alkali electrolytic bath;
(5) NaCl electrolysis: electrolyzing brine, returning chlorine to chlorohydrination and NaOH to causticization. The method achieves clean epoxypropane production with low water and energy consumption.

### CROSS-REFERENCE TO RELATED APPLICATIONS

The application claims priority to the Chinese Application No. 202411865897.4, filed on Dec. 17, 2024, the content of which is specifically and entirely incorporated herein by reference.

### FIELD

The present disclosure provides a clean production method for epoxypropane using the chlorohydrination process with a chlorine closed-loop circulation, and related to the technical field of fine chemical engineering.

### BACKGROUND

The technology of preparing epoxyalkane with a chlorohydrin method involves with preparing hypochlorous acid by using chlorine and water, and then reacting C₂-C₅ olefins with hypochlorous acid to obtain chlorohydrin, which is reacted with slaked lime or caustic soda to generate alkylene oxide, and subsequently separated with the ordinary distillation method to obtain refined alkylene oxide. The process has been industrialized as early as 1931, it has the advantages such as mature production technology, short process, large operating load flexibility, desirable selectivity, low requirements for raw material purity, safe and stable production process, small construction investment, and strong cost competitiveness of products. At present, about 40% of the epoxypropane production capacity in the world is related to the chlorohydrin method. However, the traditional chlorohydrin method device is less environmentally friendly, with large consumption of water resources and chlorine, and it is difficult to treat the causticization wastewater containing chlorides which pollute the environment, and the produced chloric acid is also seriously corrosive to the equipment. For example, the production of 1 ton of epoxypropane requires to consume about 1.5 tons of chlorine, and generates at least 40 tons of causticization wastewater containing a small amount of organic chloride and more than 2 tons of CaCl₂ waste residues.

To solve the three wastes and corrosion defects of epoxypropane produced by the chlorohydrin method, co-oxidation and direct oxidation methods have been developed in China and foreign countries. The co-oxidation method overcomes the shortcomings of the chlorohydrin method, such as high corrosiveness and large amount of waste water, but its disadvantages reside in long process flow, many raw material varieties, high propylene purity requirements, and expensive equipment costs. The epoxypropane produces a large number of co-products in the co-oxidation method. The production of 1 ton of epoxypropane will co-produce 2.5 tons of styrene or 2.4 tons of tert-butyl alcohol. The advantages of this process can only be revealed when the market demand for epoxypropane and co-products matches, In addition, the chemical oxygen demand (COD) of waste water produced by the method is high, and the treatment cost accounts for about 10% of the total investment. The advantages of the direct oxidation method are high conversion rate and selectivity, the amount of wastewater is only 30% of other existing technologies, and the energy consumption is 65% of the existing process; the process flow is simple and economical, the by-product is water, the environmental pollution is small, the land area is small, the factory infrastructure investment is small, and the investment can be reduced by 25%, but the technology is immature, the cost is high, and the safety is poor.

To this end, Dow Chemical Company of the United States of America used 10%-20% NaOH electrolyte to replace the lime milk (mainly composed of Ca(OH)₂) used in the causticization reaction, which greatly reduced the mass concentration of propanediol (PDO) and dichloropropane (DCP) in the saponification waste liquid, and obtained a relatively pure salt solution (mainly composed of NaCl and H₂O). The causticizing salt solution is refined to obtain saturated brine and sent to the electrolytic bath, where chlorine, hydrogen gas and sodium hydroxide are obtained through electrolysis of saturated brine. Chlorine is used as the reaction raw material of the chlorohydrin method, and sodium hydroxide is continued to be used to participate in the saponification reaction, which is recycled to improve the economic efficiency of the reaction. Although the improvement can effectively reduce the amount of sewage and waste residue (mainly composed of CaCl₂) generated after saponification, and decrease the pollution to soil and water sources, the refining of the causticized salt solution is too difficult to meet the requirements of electrolysis.

If the existing chlorohydrin method for producing alkylene oxides adopts NaOH causticization reaction, and can solve the problems with respect to treating the pollution of chloride-containing causticization wastewater and the recycling of concentrated brine with a lowcost, it is possible to realize closed-loop recycling of chlorine, both the cost and water consumption of epoxypropane can be greatly reduced, the chlorohydrin method for producing alkylene oxides has more development advantages than the co-oxidation method and direct oxidation method. However, the existing treatment processes of causticization waste water fail to achieve full resource utilization of causticization wastewater, thus it is urgent to develop efficient, clean and low-energy consumption processes and equipment technologies for alkylene oxide chlorohydrin method, in order to eliminate the industrial bottlenecks of high pollution and high water consumption of alkylene oxide chlorohydrin method, and improve its market competitiveness.

### SUMMARY

The present disclosure aims to overcome the defects in the prior art with respect to the technology of producing alkylene oxide with a chlorohydrin method, and provides a clean production method for epoxypropane using the chlorohydrination process with a chlorine closed-loop circulation. The chlorohydrin is rapidly causticized by a NaOH electrolytic solution, the causticization waste liquid is subjected to supercritical gasification, gas-liquid mixed phase and separation from NaCl, high-pressure lock hopper salt discharge and recycling the purified water after recovering waste heat of the high-temperature purified water, the NaCl solution diluted by the purified water is used for producing chlorine and the NaOH electrolyte is recycling, thereby overcoming the industrial bottlenecks at low cost, namely the production of epoxypropane with the chlorohydrin method involves with high water consumption, high pollution, and the full resource recycling of causticization waste liquid is difficult, and eliminating the severe corrosion of chloric acid generated by treating the causticization waste liquid to equipment, so that the method achieves a clean production of epoxy propane by using a chlorine closed-loop circulation chlorohydrin method with low water consumption and energy consumption.

The technical scheme of the present disclosure is as follows:
a clean production method for epoxypropane using the chlorohydrination process with a chlorine closed-loop circulation, the clean production method comprises the following steps: (1) a chlorohydrination process: initially dissolving chlorine in water at a temperature of 35-45° C. through a chlorine dissolver to produce hypochlorous acid and then feeding into a chlorohydrination reactor, introducing propylene through a propylene distributor according to a molar ratio of chlorine to propylene of 1:1.01-1.1, wherein a reaction pressure is normal pressure or slightly above normal pressure, a reaction temperature is within the range of 50-70° C., thereby producing a chloropropanol-containing solution having a concentration within the range of 3.0 wt %-6.0 wt %, which contains hydrochloric acid with an equal molar concentration with chloropropanol, washing a tail gas obtained from the flash evaporation process with a NaOH electrolyte, and feeding the tail gas back to a propylene recovery system; (2) a causticization process: mixing the chloropropanol-containing solution having a concentration within the range of 3.0 wt %-6.0 wt % obtained from step (1) with a NaOH electrolyte having a concentration within the range of 10 wt %-20 wt % according to a molar ratio of 1.0-1.2:1 of sodium hydroxide to the total amount of chloropropanol and hydrochloric acid, feeding the mixture to a tubular causticization reactor to perform a causticization reaction to obtain a reaction solution, wherein a causticization reaction temperature is within the range of 90-95° C., a reaction time is within the range of 0.5-2 seconds, and then feeding the reaction solution into a stripping tower to obtain a crude epoxypropane with a content within the range of 90 wt %-95 wt %, an absolute pressure of the stripping tower top is within the range of 25-65 KPa, and obtaining a causticization wastewater having a NaCl content within the range of 2.5-5.5 wt % from the stripping tower bottom; (3) a rectification process: feeding the crude epoxypropane with a content within the range of 90 wt %-95 wt % generated in the causticization process into a front distillation tower and a fractionating tower which are connected in series, the crude epoxypropane initially passes through the front distillation tower, a finished product epoxypropane is extracted from the top of the front distillation tower, high-boiling-point components are discharged from the top of the fractionating tower, both the front distillation tower and the fractionating tower are operated under normal pressure, the fractionating tower bottom temperature is within the range of 50-85° C., and the fractionating tower bottom causticization wastewater is discharged from the fractionating tower bottom; (4) a causticization wastewater treatment process: subjecting the causticization wastewater obtained from the stripping tower bottom, a tail gas washing waste liquid and a fractionating tower bottom causticization wastewater to a supercritical gasification reaction and separation by electromagnetic heating to generate a synthesis gas, a purified water and a NaCl slurry, diluting the NaCl slurry with a part of purified water to a concentrated brine with a concentration of 31.5 wt %-36.1 wt %, and then returning the concentrated brine to a chlorine-alkali electrolytic bath for recycling, and returning the remaining purified water to the chlorohydrination process; (5) a NaCl solution electrolysis process: electrolyzing the concentrated brine with a concentration of 31.5 wt %-36.1 wt % in the chlorine-alkali electrolytic bath, recycling the obtained chlorine to the chlorohydrination process for generating hypochlorous acid, and returning the NaOH electrolyte with a concentration of 10 wt %-20 wt % to the causticization process for carrying out the causticization reaction.

In the present disclosure, the type of chlorine dissolver is not particularly limited, it may be a reactor conventionally defined in the art to perform a chlorine dissolving function. Preferably, the chlorine dissolver for dissolving chlorine in water to generate hypochlorous acid is a microporous pipeline chlorine dissolver, a jet pump chlorine dissolver, or a T-type straight tube chlorine dissolver.

In the present disclosure, the type of propylene distributor is not particularly limited, it may be a reactor conventionally defined in the field that is capable of performing the propylene distribution. Preferably, the propylene distributor is a microporous pipeline mixer or a jet pump mixer, and the chlorohydrinization reactor is a pipeline reactor with an internal mixing element reinforcement, a single-tower chlorohydrin reactor, a multi-tower chlorohydrin reactor with single-tower connected in series, a tube-tower chlorohydrin reactor, or a tubular reactor.

In the present disclosure, preferably, the causticization reaction is carried out in a pipeline causticization reactor.

In the present disclosure, in order to increase the conversion rate of chloropropanol, the addition amount of NaOH is excessive. Preferably, the NaOH content in the causticizing wastewater is controlled in the range of 0.01 wt %-1.3 wt %.

In the present disclosure, preferably, the causticization wastewater obtained from the bottom of the stripping tower is pressurized to larger than or equal to 22.1 MPa by a high-pressure pump, and then subjected to a heat exchange with a mixed phase of the synthesis gas following the supercritical gasification and the supercritical high-temperature purified water; the preheated causticizing waste liquid (the causticization wastewater obtained from the stripping tower bottom, the tail gas washing waste liquid and the fractionating tower bottom causticization wastewater) is heated to a temperature of 400-700° C. by an electromagnetic heating tubular reactor for carrying out a supercritical gasification reaction, the supercritically gasified gas and liquid enter the supercritical delayed gasification reactor and stay for 1-180 minutes to generate a mixed phase of synthesis gas and supercritical high-temperature purified water, and a NaCl slurry; the extracted mixed phase of synthesis gas and supercritical high-temperature purified water performs a heat exchange with a high-pressure dissolved oxygen causticization waste liquid having a pressure larger than or equal to 22.1 MPa and then subjects to a gas-liquid separation, wherein the synthesis gas is discharged, and the purified water is recycled; the NaCl slurry is switched through the bottom lock hopper of the supercritical delayed gasification reactor, and is diluted with a part of the purified water into a concentrated brine with a concentration of 31.5 wt %-36.1 wt % and then returned to the chlorine-alkali electrolytic bath for recycling; the electromagnetic heating tubular reactor is a tubular reactor operated under the action of an electromagnetic heating regulator, when a high-frequency alternating current generates an alternating magnetic field through coils, a self-mixing enhancing internal components on the tubular reactor wall and the internal generate an eddy current for self-heating, thereby achieving the uniform heating of the high-pressure dissolved oxygen causticization waste liquid, rapid temperature rise, and gasification reaction.

In the present disclosure, the specific type of the self-mixing enhancing internal components is not particularly limited, the specific type can be selected by those skilled in the art according to actual needs. Preferably, the self-mixing enhancing internal components have a regular packing type, an X-cross sheet type, or a spiral sheet type.

In the present disclosure, preferably, a funnel-shaped liquid extraction port is arranged at the center of the supercritical delayed gasification reactor, the funnel is connected with the upper part of the supercritical delayed gasification reactor, a gas-liquid mixed phase discharge port is arranged at the top of the supercritical delayed gasification reactor, and two or more salt discharge lock hoppers are arranged at the bottom of the supercritical delayed gasification reactor.

In the present disclosure, preferably, the chlorine-alkali electrolytic bath in use is an oxygen anion membrane electrolytic bath or an anion membrane electrolytic bath.

### BRIEF DESCRIPTION OF THE DRAWINGS

FIG. 1 illustrates a schematic diagram of the process in the present disclosure.

FIG. 2 illustrates a schematic diagram of a supercritical gasification device of the present disclosure.

### DESCRIPTION OF REFERENCE SIGNS

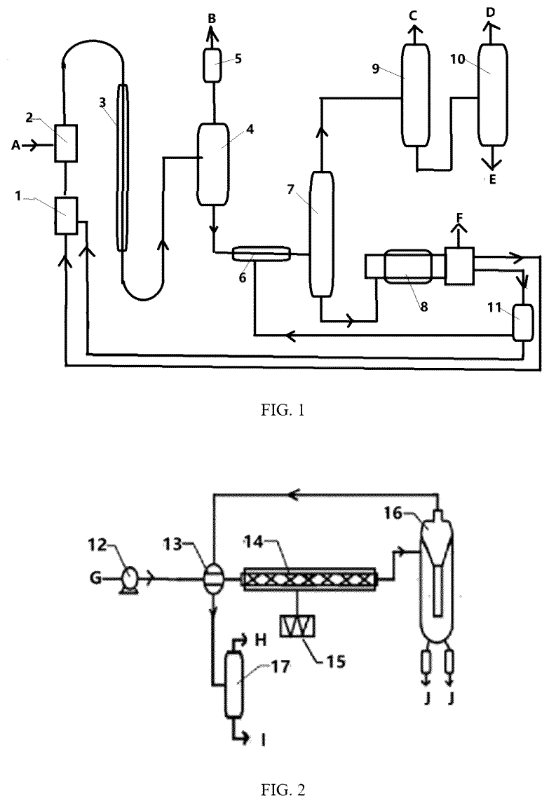

The reference signs of FIG. 1 are described as follows:
1. Chlorine dissolver
2. Propylene distributor
3. Chlorohydrination reactor
4. Flash tower
5. Alkali washing tower
6. Tubular causticization reactor
7. Stripping tower
8. Supercritical gasification device
9. Front distillation tower
10. Fractionating tower
11. Chlorine-alkali electrolytic bath
A. Propylene inlet
B. Purified tail gas outlet
C. Epoxy propane outlet
D. Heavy component outlet
E. Causticizing liquid outlet
F. Synthesis gas outlet

The reference signs of FIG. 2 are described as follows:
12. High pressure feed pump
13. Heat exchanger
14. Electromagnetic heating tubular reactor
15. Electromagnetic heating regulator
16. Supercritical delayed gasification reactor
17. Gas-liquid separator
G. NaOH causticizing waste liquid inlet
H. Synthesis gas outlet
I. Purified water outlet
J. Concentrated brine outlet

### DETAILED DESCRIPTION

The terminals and any value of the ranges disclosed herein are not limited to the precise ranges or values, such ranges or values shall be comprehended as comprising the values adjacent to the ranges or values. As for numerical ranges, the endpoint values of the various ranges, the endpoint values and the individual point values of the various ranges, and the individual point values may be combined with one another to produce one or more new numerical ranges, which should be deemed to have been specifically disclosed herein.

The present disclosure provides a clean production method for epoxypropane using the chlorohydrination process with a chlorine closed-loop circulation, the clean production method comprises the following steps: (1) a chlorohydrination process: initially dissolving chlorine in water at a temperature of 35-45° C. by a chlorine dissolver 1 to produce hypochlorous acid and then feeding into a chlorohydrination reactor 3, introducing propylene through a propylene inlet A of a propylene distributor 2 according to a molar ratio of chlorine to propylene of 1:1.01-1.1, wherein a reaction pressure is normal pressure or slightly above normal pressure, a reaction temperature is within the range of 50-70° C., thereby producing a chloropropanol-containing solution having a concentration within the range of 3.0 wt %-6.0 wt %, which contains hydrochloric acid with an equal molar concentration with chloropropanol, washing a tail gas obtained from the flash evaporation process of a flash tower 4 with a NaOH electrolyte in an alkali washing tower 5, and feeding the tail gas back to a propylene recovery system via a purified tail gas outlet B; (2) a causticization process: rapidly mixing the chloropropanol-containing solution having a concentration within the range of 3.0 wt %-6.0 wt % obtained from step (1) with a NaOH electrolyte having a concentration within the range of 10 wt %-20 wt % according to a molar ratio of 1.0-1.2:1 of sodium hydroxide to the total amount of chloropropanol and hydrochloric acid in a time of 0.5-1 second, feeding the mixture to a tubular causticization reactor 6 to perform a causticization reaction to obtain a reaction solution, wherein a causticization reaction temperature is within the range of 90-95° C., a reaction time is within the range of 0.5-2 seconds, and then feeding the reaction solution into a stripping tower 7 to obtain a crude epoxypropane with a content within the range of 90 wt %-95 wt %, an absolute pressure of the top of the stripping tower 7 is within the range of 25-65 KPa, and obtaining a causticization wastewater having a NaCl content within the range of 2.5-5.5 wt % from the bottom of the stripping tower 7; (3) a rectification process: feeding the crude epoxypropane with a content within the range of 90 wt %-95 wt % generated in the causticization process into a front distillation tower 9 and a fractionating tower 10 which are connected in series, the crude epoxypropane initially passes through the front distillation tower 9 for distillation, a finished product epoxypropane is extracted from an epoxypropane outlet C at the top of the front distillation tower 9, a bottom liquid of the front distillation tower is further delivered to the fractionating tower 10 for distillation, high-boiling-point components are discharged from a heavy component outlet D at the top of the fractionating tower 10, a fractionating tower bottom causticization wastewater is discharged from a causticizing liquid outlet E at the bottom of said fractionating tower 10; both the front distillation tower 9 and the fractionating tower 10 are operated under normal pressure, the bottom temperature of said fractionating tower 10 is within the range of 50-85° C.; (4) a causticization wastewater treatment process: subjecting the causticization wastewater obtained from the bottom of the stripping tower 7, a tail gas washing waste liquid discharged from the bottom of the alkali washing tower 5, and the fractionating tower bottom causticization wastewater to a supercritical gasification reaction and separation by electromagnetic heating in a supercritical gasification device 8 to generate a synthesis gas, a purified water, and a NaCl slurry with a concentration larger than or equal to 70%, diluting the NaCl slurry with a concentration larger than or equal to 70% by using a part of purified water to a concentrated brine with a concentration of 31.5 wt %-36.1 wt %, and then returning the concentrated brine to a chlorine-alkali electrolytic bath 11 for recycling, and returning the remaining purified water to the chlorohydrination process; (5) a NaCl solution electrolysis process: electrolyzing the concentrated brine with a concentration of 31.5 wt %-36.1 wt % in the chlorine-alkali electrolytic bath 11, recycling the obtained chlorine to the chlorohydrination process for generating hypochlorous acid, and returning the NaOH electrolyte with a concentration of 10 wt %-20 wt % to the causticization process for carrying out the rapid causticization reaction.

In the present disclosure, the type of chlorine dissolver is not particularly limited, it may be a reactor conventionally defined in the art to perform a chlorine dissolving function. Preferably, the chlorine dissolver 1 for dissolving chlorine in water to generate hypochlorous acid is a microporous pipeline chlorine dissolver, a jet pump chlorine dissolver, or a T-type straight tube chlorine dissolver.

In the present disclosure, the type of propylene distributor and the chlorohydrinization reactor are not particularly limited. Preferably, the propylene distributor 2 is a microporous pipeline mixer or a jet pump mixer, and the chlorohydrinization reactor 3 is a pipeline reactor with an internal mixing element reinforcement, a single-tower chlorohydrin reactor, a multi-tower chlorohydrin reactor with single-tower connected in series, a tube-tower chlorohydrin reactor, or a tubular reactor.

In the present disclosure, preferably, the causticization reaction is carried out in a tubular causticization reactor 6.

In the present disclosure, in order to increase the conversion rate of chloropropanol, the addition amount of NaOH is excessive. Preferably, the NaOH content in the causticizing wastewater is within the range of 0.01 wt %-1.3 wt %.

In the present disclosure, preferably, the causticization wastewater obtained from the bottom of the stripping tower 7 enters a high-pressure pump 12 through a NaOH causticizing waste liquid inlet G, and is pressurized to larger than or equal to 22.1 MPa by a high-pressure pump 12, and then subjected to a heat exchange with a mixed phase of the synthesis gas following the supercritical gasification and the supercritical high-temperature purified water via a heat exchanger 13; the preheated causticizing waste liquid (the causticization wastewater obtained from the stripping tower bottom, the tail gas washing waste liquid and the fractionating tower bottom causticization wastewater) is heated to a temperature of 400-700° C. by an electromagnetic heating tubular reactor 14 for carrying out a supercritical gasification reaction, the supercritically gasified gas and liquid enter the supercritical delayed gasification reactor 16 and stay for 1-180 minutes to generate a mixed phase of synthesis gas and supercritical high-temperature purified water, and a NaCl slurry with a high concentration; the extracted mixed phase of synthesis gas and supercritical high-temperature purified water performs a heat exchange with a high-pressure dissolved oxygen causticization waste liquid and then subjects to a gas-liquid separation, wherein the synthesis gas is discharged via a synthesis gas outlet H at the top of a gas-liquid separator 17, and the purified water separated from the bottom of the gas-liquid separator 17 is discharged via a purified water outlet I for recycling; the NaCl slurry with a high concentration is switched through the bottom lock hopper of the supercritical delayed gasification reactor 16, and is diluted with a part of the purified water into a concentrated brine with a concentration of 31.5 wt %-36.1 wt % and then returned to the chlorine-alkali electrolytic bath 11 for recycling; the electromagnetic heating tubular reactor 14 is a tubular reactor operated under the action of an electromagnetic heating regulator 15, when a high-frequency alternating current generates an alternating magnetic field through coils, a self-mixing enhancing internal components on the tubular reactor wall and the internal generate an eddy current for self-heating, thereby achieving the uniform heating of the high-pressure dissolved oxygen causticization waste liquid, rapid temperature rise, and gasification reaction.

In the present disclosure, the specific type of the self-mixing enhancing internal components is not particularly limited, the specific type can be selected by those skilled in the art according to actual needs. Preferably, the self-mixing enhancing internal components have a regular packing type, an X-cross sheet type, or a spiral sheet type.

In the present disclosure, preferably, a funnel-shaped liquid extraction port is arranged at the center of the supercritical delayed gasification reactor 16, the funnel is connected with the upper part of the supercritical delayed gasification reactor 16, a gas-liquid mixed phase discharge port is arranged at the top of the supercritical delayed gasification reactor 16, and two or more salt discharge lock hoppers are arranged at the bottom of the supercritical delayed gasification reactor 16.

In the present disclosure, preferably, the chlorine-alkali electrolytic bath 11 in use is an oxygen anion membrane electrolytic bath or an anion membrane electrolytic bath.

The present disclosure will be described in detail below with reference to examples.

The present disclosure was performed according to the schematic diagrams shown in FIG. 1 and FIG. 2, the specific reaction conditions and experimental results were as follows:

### EXAMPLE 1

A clean production method for epoxypropane using the chlorohydrination process with a chlorine closed-loop circulation, comprised the following steps:
(1) a chlorohydrination process: chlorine was initially dissolved by a chlorine dissolver 1 (a microporous pipeline chlorine dissolver) in water at a temperature of 35° C. to produce hypochlorous acid and then fed into a chlorohydrination reactor 3 (a pipeline reactor with an internal mixing element reinforcement), propylene was introduced through a propylene inlet A of a propylene distributor 2 (a microporous pipeline mixer) according to a molar ratio of chlorine to propylene of 1:1.01, wherein the reaction pressure was normal pressure, the reaction temperature was 50° C., a chloropropanol-containing solution with a concentration of 6.0 wt % was generated, which contained hydrochloric acid with an equal molar concentration with chloropropanol, the tail gas obtained from the flash evaporation process of a flash tower 4 was washed with a NaOH electrolyte in an alkali washing tower 5, and the tail gas was fed back to a propylene recovery system via a purified tail gas outlet B;
(2) a causticization process: the chloropropanol-containing solution having a concentration of 6.0 wt % obtained from step (1) was rapidly mixed with a NaOH electrolyte having a concentration of 20 wt % according to a molar ratio of 1.0:1 of sodium hydroxide to the total amount of chloropropanol and hydrochloric acid in a time of 0.6 second, the mixture was fed into a tubular causticization reactor 6 to perform a causticization reaction to obtain a reaction solution, wherein the causticization reaction temperature was 91° C., the reaction time was 0.8 second, the reaction solution was then fed into a stripping tower 7 to obtain crude epoxypropane with a content of 92 wt %, an absolute pressure of the top of the stripping tower 7 was 25 KPa, and a causticization wastewater with a NaCl content of 5.5 wt % was obtained from the bottom of the stripping tower 7;
(3) a rectification process: the crude epoxypropane with a content of 92 wt % generated in the causticization process was fed into a front distillation tower 9 and a fractionating tower 10 which were connected in series, the crude epoxypropane initially passed through the front distillation tower 9 for distillation, the finished product epoxypropane was extracted from an epoxypropane outlet C at the top of said front distillation tower 9, the bottom liquid of the front distillation tower was further delivered to the fractionating tower 10 for distillation, the high-boiling-point components were discharged via a heavy component outlet D at the top of the fractionating tower 10, the causticization wastewater was discharged through a causticizing liquid outlet E at the bottom of the fractionating tower 10; both the front distillation tower 9 and the fractionating tower 10 were operated under normal pressure, the bottom temperature of the fractionating tower was controlled at 85° C.;
(4) a causticization wastewater treatment process: the causticization wastewater obtained from the bottom of the stripping tower 7, the tail gas washing waste liquid discharged from the bottom of the alkali washing tower 5, and the causticization wastewater at the bottom of the fractionating tower 10 were mixed as the causticization waste liquid, the causticization waste liquid enter a high-pressure pump 12 through a NaOH causticizing waste liquid inlet G, and which was pressurized to 24 MPa by a high-pressure pump 12, and then subjected to a heat exchange with a mixed phase of the synthesis gas following the supercritical gasification and the supercritical high-temperature purified water by a heat exchanger 13; the preheated causticizied waste liquid was heated to a temperature of 450° C. by an electromagnetic heating tubular reactor 14 for carrying out a supercritical gasification reaction for 2 seconds, the supercritically gasified gas and liquid entered a supercritical delayed gasification reactor 16 and stayed for 10 minutes to generate a synthesis gas (discharged from a synthesis gas outlet F), a purified water, and a NaCl slurry with a concentration of 80%, the extracted mixed phase of synthesis gas and supercritical high-temperature purified water performed a heat exchange with a high-pressure dissolved oxygen causticization waste liquid and then subjected to a gas-liquid separation, wherein the synthesis gas was discharged from a synthesis gas outlet H at the top of the gas-liquid separator 17, and the purified water separated from the bottom was discharged via a purified water outlet I for recycling; the removal rate of COD in the purified water was 99.1%, the synthesis gas contained H₂ in an amount of 70 vol % and CO in an amount of 10 vol %; the NaCl slurry with a high concentration was switched through the bottom lock hopper of the supercritical delayed gasification reactor 16, and was diluted with a part of the purified water into a concentrated brine with a concentration of 32 wt % and then discharged it from the concentrated brine outlet J returned to the chlorine-alkali electrolytic bath 11 for recycling, the remaining purified water was returned to the chlorohydrination process;
(5) a NaCl solution electrolysis process: the concentrated brine with a concentration of 32 wt % was electrolyzed in the chlorine-alkali electrolytic bath 11 to generate NaOH electrolyte, H₂ and chlorine, the generated chlorine was recycled to the chlorohydrination process for generating hypochlorous acid, and the NaOH electrolyte with a concentration of 20 wt % was returned to the causticization process for carrying out the rapid causticization reaction.

### EXAMPLE 2

A clean production method for epoxypropane using the chlorohydrination process with a chlorine closed-loop circulation, comprised the following steps:
(1) a chlorohydrination process: chlorine was initially dissolved by a chlorine dissolver 1 (a jet pump chlorine dissolver) in water at a temperature of 45° C. to produce hypochlorous acid and then fed into a chlorohydrination reactor 3 (a tube-tower chlorohydrin reactor), propylene was introduced through a propylene inlet A of a propylene distributor 2 (a jet pump mixer) according to a molar ratio of chlorine to propylene of 1:1.1, wherein the reaction pressure was normal pressure, the reaction temperature was 70° C., a chloropropanol-containing solution with a concentration of 3.0 wt % was generated, which contained hydrochloric acid with an equal molar concentration with chloropropanol, the tail gas obtained from the flash evaporation process of a flash tower 4 was washed with a NaOH electrolyte in an alkali washing tower 5, and the tail gas was fed back to a propylene recovery system via a purified tail gas outlet B;
(2) a causticization process: the chloropropanol-containing solution having a concentration of 3.0 wt % obtained from step (1) was rapidly mixed with a NaOH electrolyte having a concentration of 10 wt % according to a molar ratio of 1.2:1 of sodium hydroxide to the total amount of chloropropanol and hydrochloric acid in a time of 1 second, the mixture was fed into a tubular causticization reactor 6 to perform a causticization reaction to obtain a reaction solution, wherein the causticization reaction temperature was 95° C., the reaction time was 2 seconds, the reaction solution was then fed into a stripping tower 7 to obtain crude epoxypropane with a content of 90 wt %, an absolute pressure of the top of the stripping tower 7 was 25 KPa, and a causticization wastewater with a NaCl content of 5.5 wt % was obtained from the bottom of the stripping tower 7;
(3) a rectification process: the crude epoxypropane with a content of 90 wt % generated in the causticization process was fed into a front distillation tower 9 and a fractionating tower 10 which were connected in series, the crude epoxypropane initially passed through the front distillation tower 9 for distillation, the finished product epoxypropane was extracted from an epoxypropane outlet C at the top of said front distillation tower 9, the bottom liquid of the front distillation tower was further delivered to the fractionating tower 10 for distillation, the high-boiling-point components were discharged via a heavy component outlet D at the top of the fractionating tower 10, the causticization wastewater was discharged through a causticizing liquid outlet E at the bottom of the fractionating tower 10; both the front distillation tower 9 and the fractionating tower 10 were operated under normal pressure, the bottom temperature of the fractionating tower was controlled at 75° C.;
(4) a causticization wastewater treatment process: the causticization wastewater obtained from the bottom of the stripping tower 7, the tail gas washing waste liquid discharged from the bottom of the alkali washing tower 5, and the causticization wastewater at the bottom of the fractionating tower 10 were mixed as the causticization waste liquid, the causticization waste liquid enter a high-pressure pump 12 through a NaOH causticizing waste liquid inlet G, and which was pressurized to 24 MPa by a high-pressure pump 12, and then subjected to a heat exchange with a mixed phase of the synthesis gas following the supercritical gasification and the supercritical high-temperature purified water by a heat exchanger 13; the preheated causticizied waste liquid was heated to a temperature of 550° C. by an electromagnetic heating tubular reactor 14 for carrying out a supercritical gasification reaction for 2 seconds, the supercritically gasified gas and liquid entered a supercritical delayed gasification reactor 16 and stayed for 5 minutes to generate a synthesis gas (discharged from a synthesis gas outlet F), a purified water, and a NaCl slurry with a concentration of 75%, the extracted mixed phase of synthesis gas and supercritical high-temperature purified water performed a heat exchange with a high-pressure dissolved oxygen causticization waste liquid and then subjected to a gas-liquid separation, wherein the synthesis gas was discharged from a synthesis gas outlet H at the top of the gas-liquid separator 17, and the purified water separated from the bottom was discharged via a purified water outlet I for recycling; the removal rate of COD in the purified water was 99.9%, the synthesis gas contained H₂ in an amount of 65 vol % and CO in an amount of 8 vol %; the NaCl slurry with a high concentration was switched through the bottom lock hopper of the supercritical delayed gasification reactor 16, and was diluted with a part of the purified water into a concentrated brine with a concentration of 36.1 wt % and discharged it from the concentrated brine outlet J returned to the chlorine-alkali electrolytic bath 11 for recycling, the remaining purified water was returned to the chlorohydrination process;
(5) a NaCl solution electrolysis process: the concentrated brine with a concentration of 36.1 wt % was electrolyzed in the chlorine-alkali electrolytic bath 11, the generated chlorine was recycled to the chlorohydrination process for generating hypochlorous acid, and the NaOH electrolyte with a concentration of 10 wt % was returned to the causticization process for carrying out the rapid causticization reaction.

The method is not only used for the preparation of epoxypropane by the chlorohydrination method, but also can be used for the resource-based epoxidation of olefins such as ethylene, butene and pentene.

The clean production method of epoxypropane chlorohydrin method with chlorine closed-loop circulation provided by the present disclosure has the following advantages: the circulation is ensured by causticizing with NaOH, and subjecting the causticization wastewater to a supercritical gasification treatment and then electrolysis to produce NaOH electrolyte and chlorine; in addition, the pipeline reactor strengthens the heat and mass transfer and inhibits salt precipitation scaling corrosion by means of self-mixing enhancing internal components; a supercritical delayed gasification reactor is used for improving the gasification efficiency and performing the water-gas mixed phase and salt phase separation by utilizing the supercritical water characteristics; organic matter in the causticization wastewater is completely gasified to produce synthesis gas containing more than 60 vol % of H₂, the removal rate of COD is more than 99%, and the brine concentration is flexibly regulated and controlled with low cost and energy consumption according to the needs of the electrolytic bath; the treatment cost and investment of the NaOH causticization wastewater resource are reduced by more than 70% compared with traditional treatment methods; the high water consumption and serious pollution of the chlorohydrination process are eliminated; the yield of epoxypropane is increased by 3 percentage points, the selectivity is improved by 10%, thereby achieving the closed-loop utilization of chlorine.

1. A clean production method for epoxypropane using the chlorohydrination process with a chlorine closed-loop circulation, the clean production method comprises the following steps: (1) a chlorohydrination process: initially dissolving chlorine in water at a temperature of 35-45°C through a chlorine dissolver to produce hypochlorous acid and then feeding into a chlorohydrination reactor, introducing propylene through a propylene distributor according to a molar ratio of chlorine to propylene of 1:1.01-1.1, wherein a reaction pressure is normal pressure, or above normal pressure but not higher than 10 KPa (gauge pressure), a reaction temperature is within the range of 50-70°C, thereby producing a chloropropanol-containing solution having a concentration within the range of 3.0 wt %-6.0 wt %, which contains hydrochloric acid with an equal molar concentration with chloropropanol, washing a tail gas obtained from the flash evaporation process with a NaOH electrolyte, and feeding the tail gas back to a propylene recovery system; (2) a causticization process: mixing the chloropropanol-containing solution having a concentration within the range of 3.0 wt %-6.0 wt % obtained from step (1) with a NaOH electrolyte having a concentration within the range of 10 wt %-20 wt % according to a molar ratio of 1.0-1.2:1 of sodium hydroxide to the total amount of chloropropanol and hydrochloric acid, feeding the mixture to a tubular causticization reactor to perform a causticization reaction to obtain a reaction solution, wherein a causticization reaction temperature is within the range of 90-95°C, a reaction time is within the range of 0.5-2 seconds, and then feeding the reaction solution into a stripping tower to obtain a crude epoxypropane with a content within the range of 90 wt %-95 wt %, an absolute pressure of the stripping tower top is within the range of 25-65 KPa, and obtaining a causticization wastewater having a NaCl content within the range of 2.5-5.5 wt % from the stripping tower bottom; (3) a rectification process: feeding the crude epoxypropane with a content within the range of 90 wt %-95 wt % generated in the causticization process into a front distillation tower and a fractionating tower which are connected in series, the crude epoxypropane initially passes through the front distillation tower, a finished product epoxypropane is extracted from the top of the front distillation tower, high-boiling-point components are discharged from the top of the fractionating tower, both the front distillation tower and the fractionating tower are operated under normal pressure, the fractionating tower bottom temperature is within the range of 50-85°C, and the fractionating tower bottom causticization wastewater is discharged from the fractionating tower bottom; (4) a causticization wastewater treatment process: subjecting the causticization wastewater obtained from the stripping tower bottom, a tail gas washing waste liquid and a fractionating tower bottom causticization wastewater to a supercritical gasification reaction and separation by electromagnetic heating to generate a synthesis gas, a purified water and a NaCl slurry, diluting the NaCl slurry with a part of purified water to a concentrated brine with a concentration of 31.5 wt %-36.1 wt %, and then returning the concentrated brine to a chlorine-alkali electrolytic bath for recycling, and returning the remaining purified water to the chlorohydrination process; (5) a NaCl solution electrolysis process: electrolyzing the concentrated brine with a concentration of 31.5 wt %-36.1 wt % in the chlorine-alkali electrolytic bath, recycling the obtained chlorine to the chlorohydrination process for generating hypochlorous acid, and returning the NaOH electrolyte with a concentration of 10 wt %-20 wt % to the causticization process for carrying out the causticization reaction.

2. The clean production method for epoxypropane using the chlorohydrination process with a chlorine closed-loop circulation according to claim 1, wherein the chlorine dissolver for dissolving chlorine in water to generate hypochlorous acid is a microporous pipeline chlorine dissolver, a jet pump chlorine dissolver, or a T-type straight tube chlorine dissolver.

3. The clean production method for epoxypropane using the chlorohydrination process with a chlorine closed-loop circulation according to claim 1, wherein the propylene distributor is a microporous pipeline mixer or a jet pump mixer, and the chlorohydrinization reactor is a pipeline reactor with an internal mixing element reinforcement, a single-tower chlorohydrin reactor, a multi-tower chlorohydrin reactor with single-tower connected in series, a tube-tower chlorohydrin reactor, or a tubular reactor.

4. The clean production method for epoxypropane using the chlorohydrination process with a chlorine closed-loop circulation according to claim 1, wherein the causticization reaction is carried out in a pipeline reactor, and the NaOH content in the causticizing wastewater is within the range of 0.01 wt %-1.3 wt %.

5. The clean production method for epoxypropane using the chlorohydrination process with a chlorine closed-loop circulation according to claim 1, wherein the causticization wastewater obtained from the bottom of the stripping tower is pressurized to larger than or equal to 22.1 MPa by a high-pressure pump, and then subjected to a heat exchange with a mixed phase of the synthesis gas following the supercritical gasification and the supercritical high-temperature purified water;
the preheated causticizing waste liquid is heated to a temperature of 400-700°C by an electromagnetic heating tubular reactor for carrying out a supercritical gasification reaction, the supercritically gasified gas and liquid enter the supercritical delayed gasification reactor and stay for 1-180 minutes to generate a mixed phase of synthesis gas and supercritical high-temperature purified water, and a NaCl slurry; the extracted mixed phase of synthesis gas and supercritical high-temperature purified water performs a heat exchange with a high-pressure dissolved oxygen causticization waste liquid and then subjects to a gas-liquid separation, wherein the synthesis gas is discharged, and the purified water is recycled; the NaCl slurry is switched through the bottom lock hopper of the supercritical delayed gasification reactor, and is diluted with a part of the purified water into a concentrated brine with a concentration of 31.5 wt %-36.1 wt % and then returned to the chlorine-alkali electrolytic bath for recycling; the electromagnetic heating tubular reactor is a tubular reactor operated under the action of an electromagnetic heating regulator, when a high-frequency alternating current generates an alternating magnetic field through coils, a self-mixing enhancing internal components on the tubular reactor wall and the internal generate an eddy current for self-heating, wherein the self-mixing enhancing internal components have a regular packing type, an X-cross sheet type, or a spiral sheet type; a funnel-shaped liquid extraction port is arranged at the center of the supercritical delayed gasification reactor, the funnel is connected with the upper part of the supercritical delayed gasification reactor, a gas-liquid mixed phase discharge port is arranged at the top of the supercritical delayed gasification reactor, and two or more salt discharge lock hoppers are arranged at the bottom of the supercritical delayed gasification reactor.

6. The clean production method for epoxypropane using the chlorohydrination process with a chlorine closed-loop circulation according to claim 1, wherein the chlorine-alkali electrolytic bath in use is an oxygen anion membrane electrolytic bath or an anion membrane electrolytic bath.
